(19) United States
(12) Patent Application Publication
(10) Pub. No.: US 2023/0380951 A1
(43) Pub. Date: Nov. 30, 2023

(54) DEVICES AND METHODS FOR TREATING OCCLUSIONS

(71) Applicant: W. L. Gore & Associates, Inc., Newark, DE (US)

(72) Inventors: Erin P. Childers, Flagstaff, AZ (US); Paul Morgan, Gilbert, AZ (US); Edward E. Shaw, Flagstaff, AZ (US); Maya C. Uddin, Phoenix, AZ (US); Kimberley Wunder, Phoenix, AZ (US)

(21) Appl. No.: 18/032,386

(22) PCT Filed: Oct. 18, 2021
(86) PCT No.: PCT/US2021/055399

§ 371 Date: Apr. 18, 2023

Related U.S. Application Data
(60) Provisional application No. 63/093,269, filed on Oct. 18, 2020.

(51) Int. Cl. A61F 2/07

(52) U.S. Cl. CPC A61F 2/07; A61F 2002/067

(57) ABSTRACT

A device having a support structure and a covering material, the device operable to be delivered to an at least partially occluded lumen including a non-bifurcated portion, a first bifurcated portion, and a second bifurcated portion, the device comprising a body including a primary portion, a first branch, and a second branch, the primary portion defining a primary lumen, the primary portion defined between a first open end and a flow divider, the first branch defining a first branch lumen, the first branch extending from the primary portion at the flow divider to a first branch open end, and the second branch defining a second branch lumen, the second branch extending from the primary portion at the flow divider to a second branch open end, the body having a radial wall strength sufficient to resist inward radial force and collapse of the primary, first branch, and second branch lumens.

### CROSS-REFERENCE TO RELATED APPLICATION

This application is a national phase application of PCT Application No. PCT/US2021/055399, internationally filed on Oct. 18, 2021, which claims the benefit of Provisional Application No. 63/093,269, filed Oct. 18, 2020, which are incorporated herein by reference in their entireties for all purposes.

### FIELD

The present disclosure relates generally to apparatuses, systems, and methods for treating occlusions of branched vasculature. More specifically, the disclosure relates to apparatuses, systems, and methods for implanting at a branched vessel or artery that is patent and provides blood flow through the occluded vasculature.

### BACKGROUND

Patients can develop occlusions in various parts of their vasculature. The occlusions decrease blood flow and can result in various complications including pain, loss of function of a body part, and contribution to further disease states. Treatment of various portions of the vasculature may require the installation of one or more medical devices. Installation of medical devices that effectively restore blood flow through the occluded vasculature at a bifurcated vessel or artery presents unique challenges. FIG. 1 of the present disclosure shows an exemplary branching artery that is at least partially occluded.

### SUMMARY

According to one example (“Example 1”), a device is provided having a support structure and a covering material, the device operable to be delivered to an at least partially occluded lumen including a non-bifurcated portion, a first bifurcated portion, and a second bifurcated portion, the device comprising a first elongated segment having two opposing ends and defining a first primary lumen extending therebetween, the first elongated segment operable to be positioned at least partially in the first bifurcated portion of the partially occluded lumen, and a second elongated segment having two opposing ends and defining a second primary lumen extending therebetween, the second elongated segment operable to be positioned at least partially in the second bifurcated portion of the partially occluded lumen, wherein a combined cross section of the first elongated segment and second elongated segment includes a combined cross section that is equal to or greater than an intraluminal cross section of the non-bifurcated portion of the at least partially occluded lumen, the first and second elongated segments having a radial wall strength sufficient to resist inward radial force exerted by the at least partially occluded vessel to resist collapse of the first and second primary lumens.

In a further example (“Example 2”) to Example 1, the first and second elongated segments are self-expandable.

In a further example (“Example 3”) to Example 1, the first and second elongated segments are balloon expandable.

According to one example (“Example 4”), a device is provided having a support structure and a covering material, the device operable to be delivered to an at least partially occluded lumen including a non-bifurcated portion, a first bifurcated portion, and a second bifurcated portion, the device comprising a primary elongated segment having two opposing ends and defining a primary lumen extending therebetween wherein a cross section of the primary elongated segment is equal to or greater than an intraluminal cross section of the non-bifurcated portion of the at least partially occluded lumen, a first elongated segment having two opposing ends and defining a first secondary lumen extending therebetween, the first elongated segment operable to be positioned at least partially in the first bifurcated portion of the partially occluded lumen, and a second elongated segment having two opposing ends and defining a second primary lumen extending therebetween, the second elongated segment operable to be positioned at least partially in the second bifurcated portion of the partially occluded lumen of the partially occluded lumen.

In a further example (“Example 5”) to Example 4, the primary, first, and second elongated segments are self-expandable.

In a further example (“Example 6”) to Example 5, the primary, first, and second elongated segments are balloon expandable.

According to one example (“Example 7”), a device is provided having a support structure and a covering material, the device operable to be delivered to an at least partially occluded lumen including a non-bifurcated portion, a first bifurcated portion, and a second bifurcated portion, the device comprising a body including a primary portion, a first branch, and a second branch, the primary portion defining a primary lumen, the primary portion defined between a first open end and a flow divider and having a primary portion length, the first branch defining a first branch lumen, the first branch extending from the primary portion at the flow divider to a first branch open end, the first branch having a first branch length, and the second branch defining a second branch lumen, the second branch extending from the primary portion at the flow divider to a second branch open end, the second branch having a second branch length, the body having a radial wall strength sufficient to resist inward radial force exerted by the at least partially occluded vessel to resist collapse of the primary, first branch, and second branch lumens.

In a further example (“Example 8”) to Example 7, the body is self-expandable.

In a further example (“Example 9”) to Example 7, the body is balloon expandable.

In a further example (“Example 10”) to Example 7, the body length is approximately from 2.5 to 5.5 centimeters.

In a further example (“Example 11”) to Example 10, the first and second branch lengths are approximately from 2 to 7 centimeters.

In a further example (“Example 12”) to Example 7, the body includes a diameter from 8 to 24 centimeters.

In a further example (“Example 13”) to Example 7, the first branch and the second branch include a diameter from 7 to 10 diameters

In a further example (“Example 14”) to Example 7, the device further comprises a first elongated segment having two opposing ends and defining a lumen extending therebetween, the first elongated segment operable to be positioned at least partially in the first branch lumen, and a second elongated segment having two opposing ends and defining a lumen extending therebetween, the second elongated segment operable to be positioned at least partially in the second branch lumen.

In a further example (“Example 15”) to Example 7, a ratio between a length of the first and second branch and the body is about 1:1.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an illustration of an abdominal aorta with a partial occlusion near a bifurcation or branch of the abdominal artery, according to an embodiment of the disclosure; and

FIG. 2A is an illustration of a branched stent device deployed in a bifurcated artery, according to an embodiment of the disclosure;

FIG. 2B is an illustration of components of a branched stent device for deployment in a bifurcated artery, according to an embodiment of the disclosure;

FIGS. 3A-3D are illustrations of a cross section of a branched stent device deployed in an artery, according to an embodiment of the disclosure;

FIG. 4A is an illustration of a branched stent device having a primary portion for deployment in the non-bifurcated portion of the artery and first and second portions deployed at least partially in the bifurcated portions of the artery, according to an embodiment of the disclosure;

FIG. 4B is an illustration of the components of a branched stent device having a primary and first and second portions for deployment in a bifurcated artery, according to an embodiment of the disclosure;

FIG. 5A is an illustration of a bifurcated stent graft with integral branches deployed in a bifurcated artery, according to an embodiment of the disclosure;

FIG. 5B is an illustration of a bifurcated stent graft with integral branches and first and second portions that can optionally be deployed with the bifurcated stent graft, according to an embodiment of the disclosure;

FIG. 6A is an illustration of a bifurcated stent graft with integral branches deployed in a bifurcated artery, the bifurcated stent graft having a truncated primary portion and truncated integral branches, according to an embodiment of the disclosure;

FIGS. 6B and 6C are illustrations of a bifurcated stent grafts with integral branches and first and second portions that can optionally be deployed with the bifurcated stent graft, according to an embodiment of the disclosure.

### DETAILED DESCRIPTION

### Definitions and Terminology

This disclosure is not meant to be read in a restrictive manner. For example, the terminology used in the application should be read broadly in the context of the meaning those in the field would attribute such terminology.

Persons skilled in the art will readily appreciate that various aspects of the present disclosure can be realized by any number of methods and apparatus configured to perform the intended functions. Stated differently, other methods and apparatus can be incorporated herein to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Certain relative terminology is used to indicate the relative position of components and features. For example, words such as “top”, “bottom”, “upper,” “lower,” “left,” “right,” “horizontal,” “vertical,” “upward,” and “downward” are used in a relational sense (e.g., how components or features are positioned relative to one another) and not in an absolute sense unless context dictates otherwise. Similarly, throughout this disclosure, where a process or method is shown or described, the method may be performed in any order or simultaneously, unless it is clear from the context that the method depends on certain actions being performed first.

With respect to terminology of inexactitude, the terms “about” and “approximately” may be used, in certain instances, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example.

As used herein, “couple” means join, connect, attach, adhere, affix, or bond, whether directly or indirectly, and whether permanently or temporarily.

As used herein, “medical devices” can include, for example stents, grafts, and stent-grafts, (whether single, multicomponent, bifurcated, branched, etc.), catheters, valves, and drug-delivering devices, to name just a few, that are implanted, acutely or chronically, in the vasculature or other body lumen or cavity at a treatment region.

As used herein, “leakage” means the unwanted or undesirable flow into or through a treatment region, where the flow is outside the lumen(s) or bod(ies) defined by the medical device(s), for example into or through an area such as a “gutter” located between a portion of a device and the adjacent body tissue, between two devices, or at an intersection of a portion of one or more devices and the adjacent body tissue.

As used herein, an “elliptical” shape refers to any shape that generally lacks a point where two lines, curves, or surfaces converge to form an angle. An “elliptical” shape encompasses traditional Euclidian geometric shapes such as circles and ellipses, as well as other non-angular shapes (that lack any angles), even if those shapes do not have designations common in Euclidian geometry.

As used herein, a “non-elliptical” shape refers to any shape that includes at least one point where two lines, curves, or surfaces converge to form an angle. A “non-elliptical” shape encompasses traditional Euclidian geometric shapes such as triangles, squares, and rectangles, as well as other angular shapes (that have at least one angle) such as crescents, even if those shapes do not have designations common in Euclidian geometry.

As used herein, “circumference” means the boundary line formed by an object, including, for example, an end of a stent or a stent wall at a cross section anywhere along the length of the stent. A “circumference” can include a boundary line formed by an object having any shape, including elliptical and non-elliptical shapes as defined herein, wherein the shape generally describes a line that encloses an area. A “circumference” can include a boundary line formed by an object or a cross section thereof regardless of whether the actual surface or cross section of the object described by the boundary is continuous or interrupted. For example, an open stent or an object comprising a series of separate segments that may or may not physically overlap or make contact with each other can still describe a “circumference” as used herein.

As used herein, “substantially conformable” refers to the capacity of an object to dimensionally conform to another object. The term “substantially conformable” as used herein can describe an object that is designed and given a predetermined structure and shape that fits into or against another shape, objects that have predetermined shapes that are at least in part in complementary to one another while other portions of the objects may have a shape capable of flexibly and adaptably changing to conform to another object, and objects that generally have the capacity to adapt in shape and/or conformation to other objects without any requirement for designed or predetermined complementarity to another device or object.

In various embodiments, the devices disclosed herein may comprise a covering material. A covering material may be any biocompatible or biodegradable material, as described in detail elsewhere herein. A covering material in accordance with various embodiments forms a generally continuous surface or surfaces of a component of the device, defining a lumen and an outer surface of the component of the device. The covering material need not be completely continuous, but may be interrupted by openings at the ends of the elongated segments or branch segments, open stent regions, and/or fenestrations such as side branch openings. The covering material may be applied to the device by any of a variety of methods, including, for example, wrapping, forming, or molding a covering material about a mandrel.

In accordance with various embodiments, a device may comprise such features as radiopaque markers or similar features that aid visualization of the device within the body during deployment and positioning.

In various embodiments, a device may comprise coatings. The coatings of the device components may be in contact with other objects including other devices or device components or interior surfaces of the vasculature.

In various embodiments, the devices disclosed herein may comprise a support structure (e.g., a stent of any suitable configuration). The support structure may be any suitable material including, for example, stainless steel, nitinol, and the like. The support structure may comprise a plurality of stent rings. The stent rings may be operatively coupled to one another with a wire. A wire used to couple stent rings may attach to the peak of a first stent ring and a valley of a second stent ring. The stent ring may be arranged such that the peaks in valleys are in-phase (e.g., the peaks first stent ring share a common centerline with the peaks of the second stent) or out of phase (e.g., the peaks of the first stent ring share a common centerline with the valleys of the second stent ring).

A device in accordance with various embodiments can comprise a first and a second elongated segment, each having two opposing ends and each defining a lumen extending between the ends. The lumens defined by the elongated segments are referred to as primary lumens. Each elongated segment may be comprised of two or more separate subsegments that are joined to form a single elongated segment, as described herein, with the single elongated segment comprised of two or more separate subsegments defining a single lumen and having two opposing ends. Furthermore, any use of the term “elongated segment” in the present disclosure can also include “subsegment.” In accordance with various embodiments, the device can comprise two or more elongated segments.

### DESCRIPTION OF VARIOUS EMBODIMENTS

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting. Furthermore, although the following may include discussion of specific vasculature, such as the aorta or iliac arteries, it is within the scope of this disclosure that the disclosed devices may be implemented within any applicable vasculature of a patient, and more specifically within any bifurcated arteries of veins.

The present disclosure relates to a number of non-limiting embodiments, each of which may be used alone or in coordination with one another. A device in accordance with various embodiments can be any suitable medical device or devices installable within the vasculature or other body lumens and configured to provide for isolation of a treatment region from fluid pressure. In various embodiments, a device can comprise one or more elongated segments that approximate the cross-sectional profile of the vasculature when implanted in a treatment region.

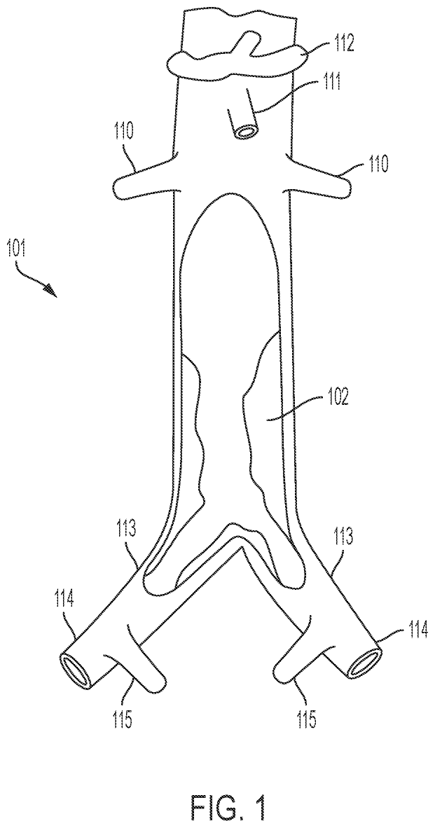

For example, FIG. 1 illustrates a vasculature into which a device according to various embodiments may be implanted. The vasculature includes an abdominal aorta 101 with major branch arteries, including the renal arteries 110, the superior mesenteric artery (“SMA”) 111, the celiac artery 112, the common iliac arteries 113, the external iliac arteries 114, and the internal iliac arteries 115. In the illustrated the example, the abdominal aorta has an occlusion 202 at least partially occluding the abdominal aorta 101.

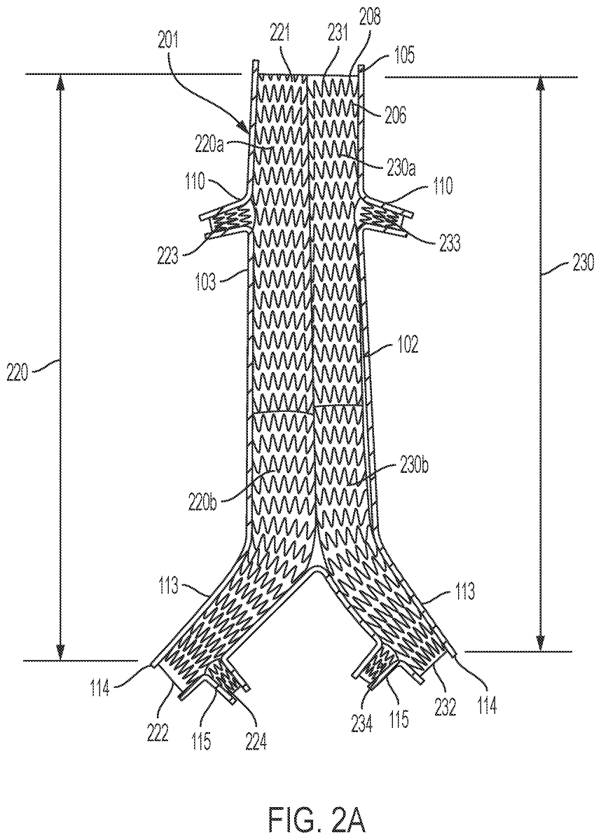

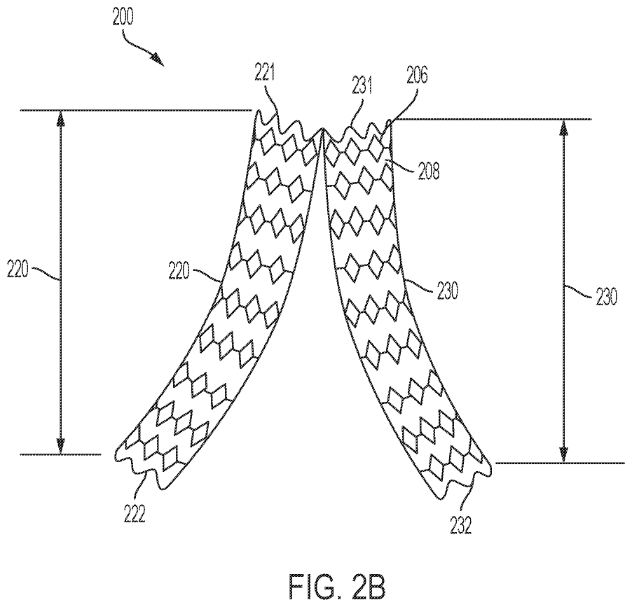

Referring to FIG. 2A, a branched stent device 200 is shown positioned in the vasculature of a patient, the branched stent device 200 comprising two or more elongated segments, such as a first elongated segment 220 and a second elongated segment 230. Each of the elongated segments 220, 230 may comprise a frame 206 and a covering 208. The frame 206 supports the covering 208. First elongated segment 220 can be comprised of subsegment 220a and subsegment 220b, and second elongated segment can be comprised of subsegment 230a and subsegment 230b. First elongated segment 220 can have a proximally oriented first end 221 and a second end 222, and likewise, second elongated segment 230 can have a proximally oriented first end 231 and a second end 232. The elongated segments can be deployed at a treatment site in a vasculature 101 such as an abdominal aorta that has an at least partial occlusion 102 (see also FIG. 1) or other body lumen in any suitable configuration. For example, the elongated segments can be installed in a configuration to conduct blood or other bodily fluids between a proximal aortic lumen 105 and distal lumens such as those of the common iliac arteries 113 and/or one or more side branch vessels such as the renal arteries 110 and the internal iliac arteries 115. In the illustrated example, subsegments 220a and 230a of first and second elongated segments 220 and 230 of the device are implanted in the proximal portion of the treatment region to receive blood from proximal aortic lumen 105 and perfuse renal arteries 110 via branch first branch segment 223 and third branch segment 233, and subsegments 220b and 230b of the device conduct blood distally to the external iliac arteries 114 at second ends 222 and 232 of the first and second elongated segments 220 and 230, as well as to internal iliac arteries 115 via second branch segment 224 and fourth branch segment 234. In various other embodiments, the second ends of elongated segments can be located in other portions of a treatment region, for example, in the common iliac arteries 113 or in a region of normal aorta distal to the occlusion 102. In accordance with various embodiments, first ends 221 and 231 and second ends 222 and 232 of first elongated segment 220 and second elongated segment 230 may be located in any suitable portion of a treatment region.

In various embodiments, one or more elongated segments may be joined to another medical device. For example, a device comprising two elongated segments, similar to subsegments 220a and 230a, as illustrated in FIG. 2, can be joined at the second ends of the elongated segments to a proximal end of a bifurcated stent-graft, wherein the bifurcated stent-graft functions to deliver blood to the distal portion of the treatment region. The device comprising two elongated segments can be joined to the bifurcated stent-graft in a substantially fluid-tight manner during deployment of the elongated segments. In this manner, a device in accordance with various embodiments comprising two or more elongated segments and having branch segments, as described below, can be deployed in a proximal portion of a treatment region, such as a proximal aorta having renal artery branches, and joined to a second medical device, for example, a bifurcated stent-graft suitable for installation in a distal portion of a treatment region such as the distal portion of an occlusion and the common iliac arteries. Any combination of a device in accordance with various embodiments deployed in any portion of a treatment region and joined with any other medical device is within the scope of the present disclosure.

In accordance with various embodiments, a first elongated segment and a second elongated segment have a combined cross section that is substantially conformable to an intraluminal cross section of a body lumen. For example, in any portion of vasculature 101 where first elongated segment 220 and second elongated segment 230 occupy the same cross-sectional profile (e.g., the infrarenal aortic neck 203 or the location of first end 221 of the first elongated segment 220 and first end 231 of the second elongated segment 230 in a proximal aortic lumen 105 as illustrated here), first elongated segment 220 and second elongated segment 230 are substantially conformable to the intraluminal cross section of the vasculature. The substantially conformable cross-sectional profiles of the first elongated segment 220 and the second elongated segment 230 have a combined cross section that approximates the intraluminal cross-sectional profile of vasculature 101. The substantially conformable character of the first elongated segment and the second elongated segment to the intraluminal cross section of the vasculature at a cross section can contribute promoting more desirable flow characteristics in the treatment region such as un-obstructed flow, evenly distributed flow, steady flow or flow that is otherwise consistent with flow through a healthy body lumen.

In these embodiments, first elongated segment 220 can have any suitable shape. Similarly, second elongated segment 230 can have any suitable shape that is complementary to the shape of first elongated segment 220. This complementary arrangement occurs where the combined cross-sectional profile of first elongated segment 220 and second elongated segment 230, when installed in vasculature 101, substantially approximates the intraluminal cross-sectional profile of vasculature 101 to minimize leakage and improve fluid flow characteristics at the treatment site. For example, first end 221 of first elongated segment 220 can have a substantially elliptical cross-sectional profile when installed at the treatment region corresponding to proximal lumen 105. First end 231 of second elongated segment 230 can have a suitably complementary substantially elliptical cross-sectional profile at the end installed at the treatment region corresponding to proximal lumen 105, where first elongated segment 220 and second elongated segment 230 are installed together. In this embodiment, each of the first end 221 of the first elongated segment 220 and the first end 231 of the second elongated segment 230 is installed on substantially the same level or cross-sectional plane of the vasculature, though in other embodiments they can be installed in other planes or in a longitudinally displaced relationship. Moreover, because of the complementary shape of the each of the ends, the combined profile of the ends forms a generally elliptical cross-section that approximates the generally elliptical cross-section of vasculature 101. The substantial conformation of the first elongated segment 220 and the second elongated segment 230 to the intraluminal cross section of proximal lumen 105 allows blood and other bodily fluids to flow through the lumens of the elongated segments approximating vasculature 101.

In various embodiments, the first elongated segment and the second elongated segment can be of any suitable size and shape to provide a combined cross section that is substantially conformable to an intraluminal cross section of a body lumen. The first and second elongated segments can be of sizes and shapes that are complementary to one another and together provide a combined cross section, such as an ellipse, that generally approximates the size and shape of a body lumen and substantially conforms to the intraluminal cross section of a body lumen when deployed together within the lumen.

FIG. 2B illustrates the branched stent device 200 wherein the first elongated segment 220 and the second elongated segment 230 do not each include subsections.

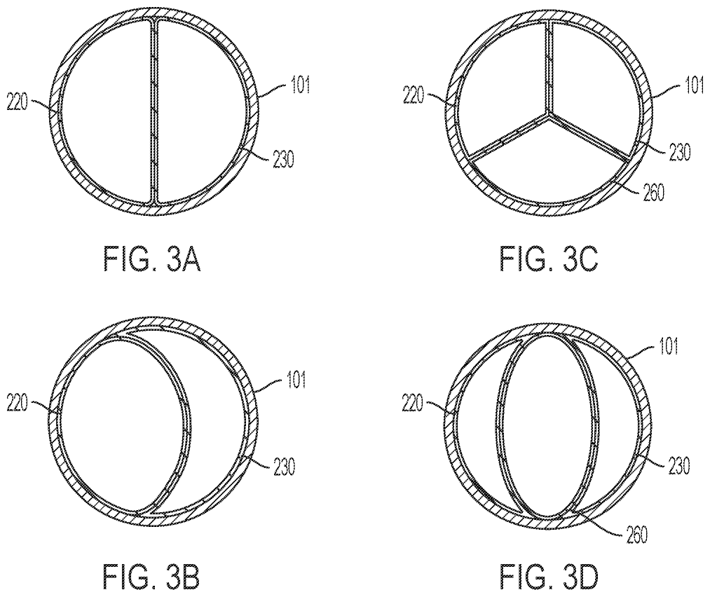

For example, and with reference to FIG. 3A, first elongated segment 220 and second elongated segment 230 can both have generally elliptical cross sections that are complementary to one another such that the combined cross section of the elongated segments substantially conform to the intraluminal cross section of vasculature 101. In various other embodiments and with reference to FIG. 3B, first elongated segment 220 can have a cross-sectional profile that is generally elliptical as illustrated in FIG. 3B, while second elongated segment 230 can a shape that is complementary to the cross section or a portion of the cross section of the first elongated segment 220, such as a crescent shape with an interior arc that complements the elliptical profile of the first elongated segment 220. In accordance with various embodiments, the combined cross-sectional profile of first elongated segment 220 and second elongated segment 230 is generally elliptical and approximates the intraluminal cross section of vasculature 201 regardless of the individual cross-sectional profiles of the component elongated segments.

In various embodiments, a device can comprise three or more elongated segments. As for the embodiments described above and as illustrated in FIGS. 3C and 3D, the three or more elongated segments can have shapes that are complementary to one another such that a combined cross section of the elongated segments is substantially conformable to an intraluminal cross section of a body lumen such as an ellipse. For example, each of first elongated segment 220, second elongated segment 230, and third elongated segment 260 can be generally pie-shaped, as illustrated in FIG. 3C. In this configuration, a flat portion of each pie-shaped profile is configured to abut another flat portion of a pie-shaped profile. The curved portion of each pie-shaped profile is configured to approximate a portion of vasculature 201. Other combinations of three or more elongated segments with various complementary cross-sectional profiles, such as a third elongated segment 260 with an elliptical cross section combined with crescent-shaped first elongated segment 220 and second elongated segment 230, as illustrated in FIG. 3D, are also within the scope of the present disclosure. Any number of elongated segments having any combination of cross-sectional profiles that, when installed together, form a combined cross section that is generally elliptical and/or substantially conforms to an intraluminal cross section of a body lumen is within the scope of the present disclosure.

In various embodiments, elongated segments of a device can have cross-sectional profiles that are shaped or formed prior to deployment of the elongated segments, such that the elongated segments take on a predetermined cross-sectional profile upon deployment. For example, elongated segments can be shaped or formed with cross-sectional profiles that are complementary to each other. The elongated segments can be constrained to another cross-sectional profile prior to deployment for insertion and deployment, and upon deployment, the elongated segments can take on their predetermined, complementary cross-sectional profiles that substantially conform to an intraluminal cross section a body lumen.

In various other embodiments, the cross-sectional profile of an individual elongated segment can be determined during deployment, such as by the cross-sectional profile of a balloon expansion device used in deployment. For example, an elongated segment can be plastically deformable, such that it can take on and retain the cross-sectional profile of the balloon expansion devices used to expand and deploy the elongated segment to the implanted state. Balloon expansion devices can be used that are capable of expanding an elongated segment to any suitable size and/or cross-sectional profile, such as circular, elliptical, crescent, pie-shaped or other cross-sectional profiles, such that one or more elongated segments are complementary to one another and substantially conform to the intraluminal cross section of the body lumen in which they are deployed.

In some embodiments, the elongated segments are self-expanding. The elongated segments include sufficient radial strength to expand to a predetermined diameter. More specifically, the elongated segments are operable to expand to a predetermined diameter that is sufficient for providing a cross-section in the vasculature to allow for sufficient fluid (e.g., blood) flow through the segments. Furthermore, the radial strength of the elongated segments is sufficient to limit collapse of the elongated segments within the vasculature, e.g., vasculature with occlusions.

In accordance with still other embodiments, the elongated segments can be flexible such that they can accommodate a broad range of cross-sectional profiles and conform in their individual cross-sectional profiles to the intraluminal cross section of the body lumen in which they are deployed. In these embodiments, the intraluminal cross section of the body lumen in which an elongated segment is deployed may be determined by another elongated segment and/or other medical device, either temporary or implanted, during deployment of the flexible elongated segment in the body lumen. Stated differently, the flexible elongated segment may generally lack a predetermined deployed cross-sectional profile, and the cross-sectional profile of the flexible elongated segment is determined by the cross-sectional profile of the body lumen in which it is deployed and any other elongated segments or medical devices that may be deployed therein, regardless of the cross-sectional profile of the body lumen or of those elongated segments or medical devices within the body lumen.

In accordance with various embodiments, one of the elongated segments may have the property of being flexibly able to adapt to the cross-sectional profile of the lumen in which it is located. In various other embodiments, more than one of the elongated segments may be so flexibly adaptable. For example, in a case where two flexibly adaptable elongated segments are deployed together in a body lumen, the two elongated segments would together substantially conform to one another and to the intraluminal cross section of the body lumen in which they are located. In such an embodiment, predetermined complementary cross-sectional profiles for the elongated segments are not required. These embodiments may provide advantages such as the ability to independently position an elongated segment longitudinally and/or rotationally. For example, the absence of predetermined complementarity of one elongated segment with a second elongated segment eliminates the requirement that the two complementary elongated segments be aligned longitudinally and rotationally so as to provide the planned complementary cross-sectional profile.

In accordance with any of the various embodiments described herein, the elongated segments might only be substantially conformable to the intraluminal cross section of a body lumen where there are two or more elongated segments present in the intraluminal cross section of the body lumen. Stated differently, a device in accordance with various embodiments may or may not substantially conform to the intraluminal cross section of a body lumen in cross sections in which only a single elongated segment is located. For example, a device in accordance with various embodiments can comprise two elongated segments of the same length but that are longitudinally displaced from one another within the body lumen, such that only one elongated segment is located at various cross sections within the body lumen. In this example, at the intraluminal cross section(s) of the body lumen that is occupied by a single elongated segment, the elongated segment may not substantially conform to the intraluminal cross section of the body lumen but may only partially occupy the intraluminal cross section.

In accordance with various embodiments, an elongated segment can comprise an open stent region. An elongated segment can comprise an open stent region in any portion of the elongated segment. An open stent region of an elongated segment is a portion of an elongated segment comprising support elements but lacking a covering material or otherwise having a configuration that is perfusable by a fluid. An open stent region of an elongated segment can be located at any part of an elongated segment and can comprise any portion of the elongated segment. For example, an open stent region can be located at an end of an elongated segment or anywhere along the length of the elongated segment. The open stent portion can include the entire circumference of a portion of the length of an elongated segment, or can comprise a portion of the circumference and the length of the elongated segment, forming an open stent window in an area of the elongated segment.

Each of the first, second, and/or third elongated segments 220, 230, 260 may be from about five (5) to about 15 millimeters in diameter. More specifically, the first, second, and/or third elongated segments 220, 230, 260 may be five (5), six (6), seven (7), eight (8), nine (9), 10, 11, 12, 13, 14, 15, or 16 millimeters in diameter. The overall length of the branched stent device 200 may be from about 15 millimeters to about 80 millimeters in length. The sheath size for the branched stent device 200 may be from about seven (7) Fr to about eight (8) Fr.

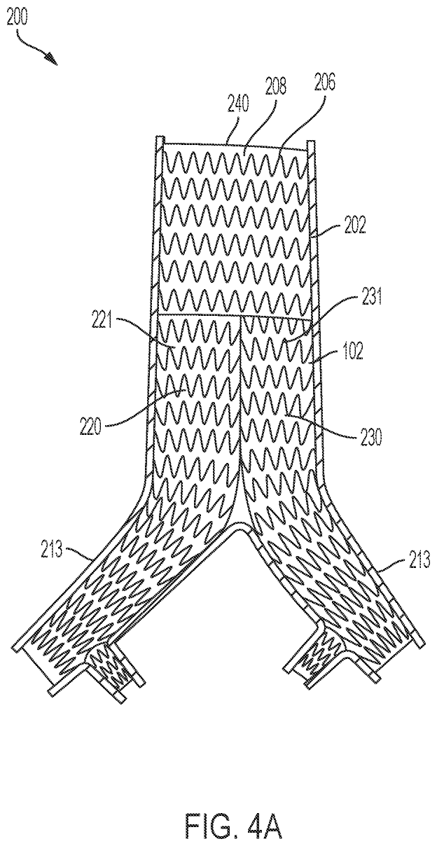

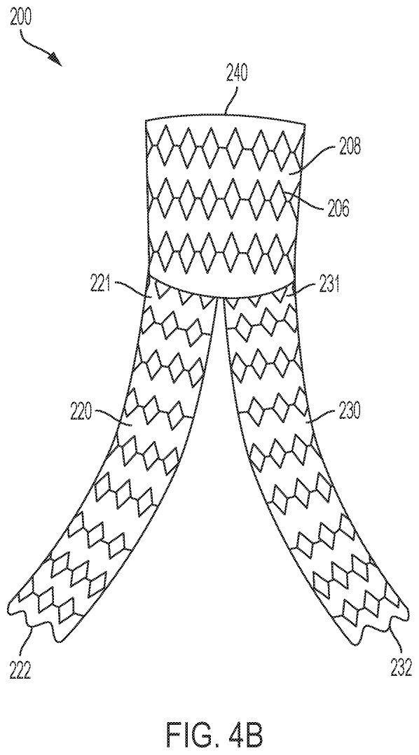

Referring now to FIG. 4A, the branched stent device 200 includes a primary stent graft 240. The primary stent graft 240 is operable to be positioned at treatment site in a vasculature 201 at the non-bifurcated portion of the treatment site. The primary stent graft 240 is sized appropriately for being positioned at the treatment site. The primary stent graft 240 is operable to receive at least portion of the first elongated segment 220 and a second elongated segment 230. For example, the proximally oriented first end 221 of the first elongated segment 220 and the proximally oriented first end 231 of the second elongated segment 230 can be positioned in the primary stent graft 240. When the first elongated segment 220 and the second elongated segment 230 may be substantially sealed with the primary segment 240 such that fluid flows into the primary segment 240 and into each of the first elongated segment 220 and the second elongated segment 230. In other embodiments, the first elongated segment 220 and the second elongated segment 230 are expanded to respective predetermined diameters, but may not necessarily form a full fluidic seal with the primary segment 240 about the interior circumference. FIG. 4B is another embodiment in which the frame includes a diamond design. It is within the scope of this disclosure to implement other appropriate frame designs. The frames may be either self-expanding or balloon expandable.

The primary stent graft 240 may be from about 18 to about 30 millimeters in diameter. The length of the primary stent graft 240 may be from about two (2) to about three (3) millimeters in length. The sheath size for the branched stent device 200 may be from about fourteen 14 Fr to about 17 Fr.

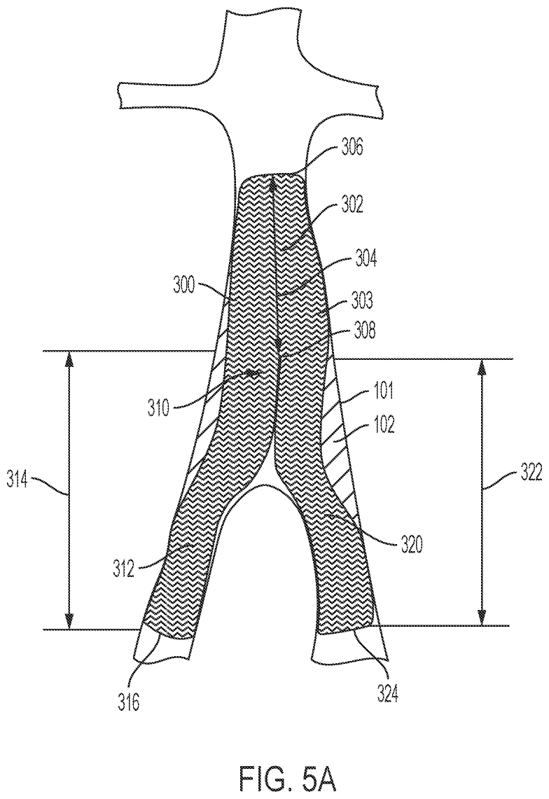

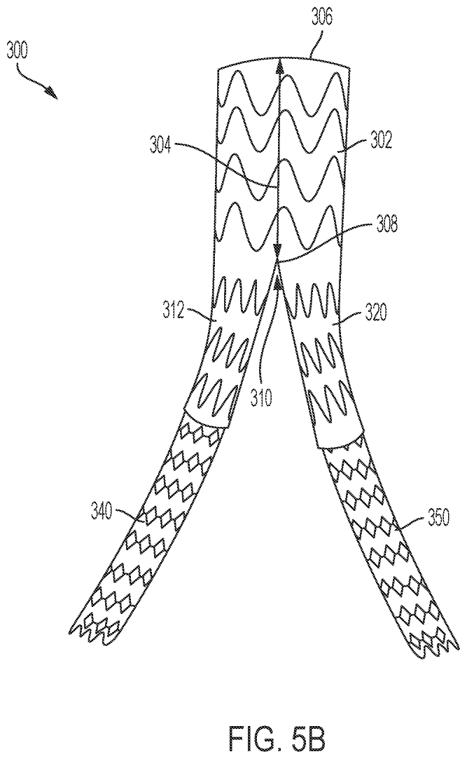

Referring now to FIG. 5A, an exemplary bifurcated stent graft 300 with integral branches is configured in the bifurcated vascular lumen. The bifurcated stent graft 300 has a primary body 302 that is a single tubular graft 303 having a length 304 from a first end 306 to the flow divider 308, where the graft bifurcation 310 starts. The bifurcated stent graft 300 comprises an integral ipsilateral branch 312 having a length 314 from the graft bifurcation 310 to the second end 316. The bifurcated stent graft 300 has an integral contralateral branch 320 having a length 322 from the graft bifurcation 310 to the second end 324 of the contralateral graft branch. In some embodiments, the bifurcated stent graft 300 may comprise an opening, or a contralateral branch with a short length for receiving the contralateral limb and may have substantially no contralateral branch. The distal end 306 of the primary body 302 is secured in the non-bifurcated portion of the vasculature and the integral ipsilateral limb is configured within one of the branches of the bifurcated vasculature. The bifurcated stent graft 300 has a lumen that extends from the distal end of the primary body 302 down into the two separate lumens after the graft bifurcation 310.

Similarly as discussed with respect to the branched stent device 200, in some embodiments, the primary body 302 and branches 312, 320 of the bifurcated stent graft 300 are self-expanding. The primary body 302 and branches 312, 320 include sufficient radial strength to expand to a predetermined diameter. More specifically, the elongated segments are operable to expand to a predetermined diameter that is sufficient for providing a cross-section in the vasculature to allow for sufficient blood flow through the segments. Furthermore, the radial strength of the elongated segments is sufficient to limit collapse of the elongated segments within the vasculature (e.g., vasculature with occlusions).

In other embodiments, the primary body 302 and branches 312, 320 of the bifurcated stent graft 300 are balloon expandable. The cross-sectional profile of an individual elongated segment can be determined during deployment, such as by the cross-sectional profile of a balloon expansion device used in deployment. For example, an elongated segment can be plastically deformable, such that it can take on and retain the cross-sectional profile of the balloon expansion devices used to expand and deploy the elongated segment to the implanted state. Balloon expansion devices can be used that are capable of expanding an elongated segment to any suitable size and/or cross-sectional profile, such as circular, elliptical, crescent, pie-shaped or other cross-sectional profiles, such that one or more elongated segments are complementary to one another and substantially conform to the intraluminal cross section of the body lumen in which they are deployed.

The primary body 302 may be from about 20 to about 23 millimeters in diameter. The length of the primary body 302 may be from about two (2) to about six (6) millimeters in length. More specifically, primary body 302 may be three (3), four (4), or 5.5 millimeters in length. The sheath size for the branched stent device 200 may be from about fourteen 14 Fr to about 17 Fr. The branches 312, 320 may be from about 10 to about 20 millimeters in diameter, and more specifically about 13 millimeters in diameter.

FIG. 5B is another embodiment in which the frame includes a diamond design. It is within the scope of this disclosure to implement other appropriate frame designs. Furthermore, the bifurcated stent graft 300 may include two or more elongated segments, such as a first elongated segment 340 and a second elongated segment 350. In some examples, the primary body 302 may and branches 312, 320 may be self-expanding and the first elongated segment 340 and the second elongated segment 350 may be balloon expandable. In other examples, the primary body 302 may and branches 312, 320 may be balloon expandable and the first elongated segment 340 and the second elongated segment 350 may be self-expanding. This allows a surgeon to select the appropriate components of the bifurcated stent graft 300 for effectively restoring flow through the vasculature, the components being selected based on the specific conditions of the occluded vasculature.

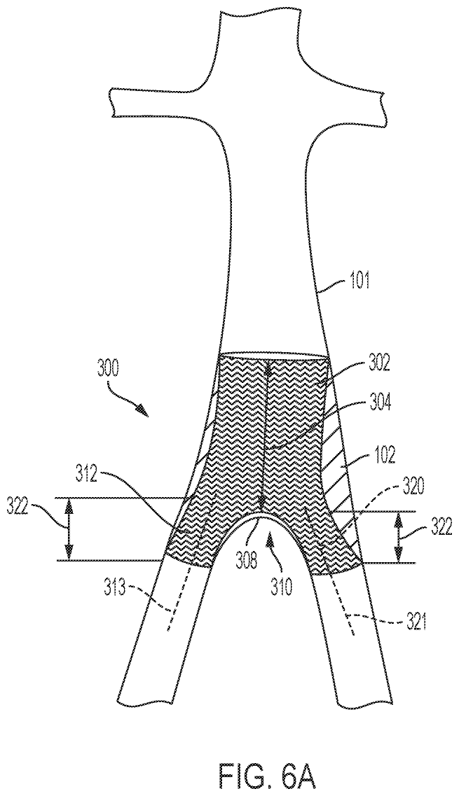

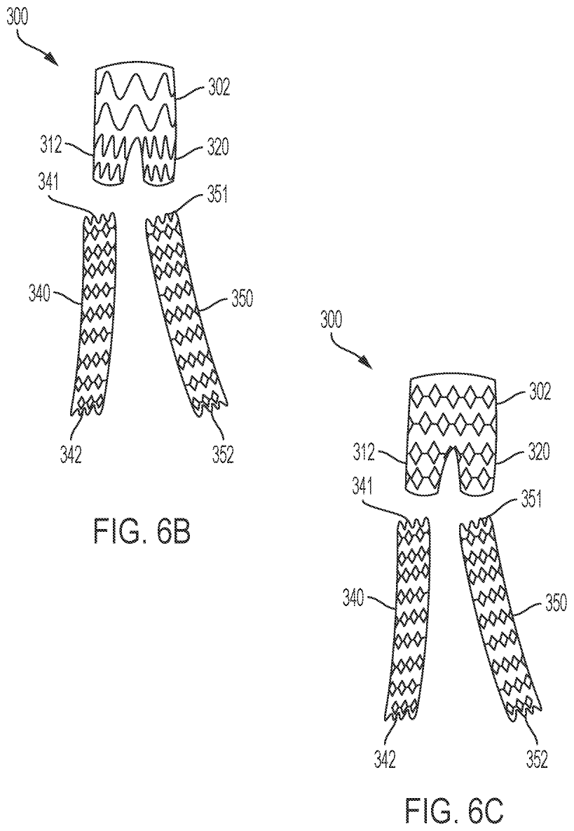

Referring now to FIG. 6A, the bifurcated stent graft 300 is provided with a primary body 302 having a length 304 from the distal end 306 to the flow divider 308 that is less than four (4) centimeters. In some embodiments, the length 304 of the primary body 302 is from about one (1) to about four (4) centimeters. In other embodiments, the length 304 of the primary body 302 is from about two (2) to about three (3) centimeters. More specifically, the length 304 of the primary body 302 is approximately 2.0, 2.5, 3.0, 3.5, or 4.0 millimeters. The length 304 of the primary body 302 may be limited to the above discussed dimensions in order to limit the chances of the primary body 302 from covering branches or access points with the bifurcated stent graft 300. The diameter of the primary body 302 is from about eight (8) to about 24 millimeters.

The branches 312, 320 extending from the primary body 302 may be at least two (2) centimeters. In some embodiments, the length 314, 322 of the primary body 302 is between two (2) and four (4) centimeters. In other embodiments, the length 304 of the primary body is between two (2) and three (3) centimeters. The length 304 of the primary body 302 may be limited to the above discussed dimensions in order to limit the chances of the primary body 302 from covering branches or access points with the bifurcated stent graft 300. The diameter of the branches 312, 320 is from about seven (7) to about 10 millimeters. In some embodiments, the ratio between the length of the primary body 302 and the branches may be about from about 1:0.75 to about 1.25:1. In some embodiments, the ratio between the length of the primary body 302 and the branches may be about 1:1.

With further reference to the branches 312, 320, each branch 312, 320 may extend from the primary body 302 at a predetermined position and angle. For example, the first branch 312 and second branch 320 each defines a first longitudinal axis 313 and a second longitudinal axis 321. The first and second branches 312, 320 extend from the primary body 302 such that an angle greater than zero is formed between the first longitudinal axis 313 and the second longitudinal axis 321. The angle formed between the first longitudinal axis 313 and the second longitudinal axis 321 may be from about 0.5 to about 30.0 degrees. In some embodiments, the first longitudinal axis 313 and the second longitudinal axis 321 are parallel to each other. In this embodiment, bases of the first and second branches 312, 320 are laterally spaced from each other to maintain separate lumens.

Referring now to FIGS. 6B and 6C, the bifurcated stent graft 300 may further include two or more elongated segments, such as a first elongated segment 340 and a second elongated segment 350. The first elongated segment 340 can have a proximally oriented first end 341 and a distally oriented second end 342, and likewise, the second elongated segment 350 can have a proximally oriented first end 351 and a distally oriented second end 352. The elongated segments 340, 350 can be deployed such that the first ends 341, 351 are positioned against the branches 312, 320. The elongated segments 340, 350 extend from the branches 312, 320 such that the second ends 342, 352 extend away from the primary body 302. In some embodiments, the elongated segments 340, 350 are positioned at least partially or fully within the branched portions of the vasculature. By including elongated segments 340, 350 that are separate from the primary body 302 and the branches 312, 320 of the bifurcated stent graft 300, the physician may implement any length, type, configuration, or diameter of elongated segments 340, 350 for the specific conditions in which the bifurcated stent graft 300 is being implanted.

Many graft materials are known, particularly known are those that can be used as vascular graft materials. In one embodiment, the materials can be used in combination and assembled together to comprise a graft. The graft materials, used in a stent-graft, can be extruded, coated or formed from wrapped films, or a combination thereof. Polymers, biodegradable and natural materials can be used for specific applications.

Examples of synthetic polymers include, but are not limited to nylon, polyacrylamide, polycarbonate, polyformaldehyde, polymethylmethacrylate, polytetrafluoroethylene, polytrifluorochlorethylene, polyvinylchloride, polyurethane, elastomeric organosilicon polymers, polyethylene, polypropylene, polyurethane, polyglycolic acid, polyesters, polyamides, their mixtures, blends and copolymers are suitable as a graft material. In one embodiment, the graft is made from a class of polyesters such as polyethylene terephthalate including DACRON® and MYLAR® and polyaramids such as KEVLAR®, polyfluorocarbons such as polytetrafluoroethylene (PTFE) with and without copolymerized hexafluoropropylene (TEFLON® or GORE-TEX®), and porous or nonporous polyurethanes. In another embodiment, the graft comprises expanded fluorocarbon polymers (especially PTFE) materials. Included in the class of preferred fluoropolymers are polytetrafluoroethylene (PTFE), fluorinated ethylene propylene (FEP), copolymers of tetrafluoroethylene (TFE) and perfluoro (propyl vinyl ether) (PFA), homopolymers of polychlorotrifluoroethylene (PCTFE), and its copolymers with TFE, ethylenechlorotrifluoroethylene (ECTFE), copolymers of ethylene-tetrafluoroethylene (ETFE), polyvinylidene fluoride (PVDF), and polyvinyfluoride (PVF). Especially preferred, because of its widespread use in vascular prostheses, is ePTFE. In another embodiment, the graft comprises a combination of the materials listed above. In another embodiment, the graft is substantially impermeable to bodily fluids. The substantially impermeable graft can be made from materials that are substantially impermeable to bodily fluids or can be constructed from permeable materials treated or manufactured to be substantially impermeable to bodily fluids (e.g. by layering different types of materials described above or known in the art). In one embodiment, the primary body and branch members, as described above, are made from any combinations of the materials above. In another embodiment, the primary body and branch members, as described above, comprise ePTFE. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers.

The stents, as described above, may be generally cylindrical when restrained and/or when unrestrained and comprise helically arranged undulations having plurality of helical turns. The undulations preferably are aligned so that they are “in-phase” with each other. More specifically, undulations comprise apices in opposing first and second directions. When the undulations are in-phase, apices in adjacent helical turns are aligned so that apices can be displaced into respective apices of a corresponding undulation in an adjacent helical turn. In one embodiment, the undulations have a sinusoidal shape. In another embodiment, the undulations are U shaped. In another embodiment, the undulations are V shaped. In another embodiment, the undulations are ovaloid shaped. These shapes are fully described in U.S. Pat. No. 6,042,605 by Gerald Martin, filed on Jul. 18, 1997, which is incorporated by reference herein in its entirety for all purposes. U.S. Pat. No. 10,299,948 by Jane Bohn, filed Nov. 24, 2015, is likewise incorporated by reference herein in its entirety for all purposes.

In another embodiment, the stents, as described above, may also be provided in the form of a series of rings arranged generally coaxially along the graft body.

In various embodiments, the stent can be fabricated from a variety of biocompatible materials including commonly known materials (or combinations of materials) used in the manufacture of implantable medical devices. Typical materials include 316L stainless steel, cobalt-chromium-nickel-molybdenum iron alloy (“cobalt-chromium”), other cobalt alloys such as L605, tantalum, nitinol, polymers, MP35N steel, polymeric materials, Pyhnox, Elgiloy, or any other appropriate biocompatible material, and combinations thereof. In one embodiment, any stent-graft described herein is a balloon expandable stent-graft. In another embodiment, any stent-graft described herein is a self-expanding stent-graft. In another embodiment, the stent is a wire wound stent. In another embodiment, the wire wound stent comprise undulations. The super-elastic properties and softness of nitinol may enhance the conformability of the stent. In addition, nitinol can be shape-set into a desired shape. That is, nitinol can be shape-set so that the frame tends to self-expand into a desired shape when the frame is unconstrained, such as when the frame is deployed out from a delivery system.

Any of a variety of bio-active agents may be implemented with any of the foregoing. For example, any one or more of (including portions thereof) the devices may comprise a bio-active agent. Bio-active agents can be coated onto one or more of the foregoing features for controlled release of the agents once the devices are implanted. Such bio-active agents can include, but are not limited to, thrombogenic agents such as, but not limited to, heparin. Bio-active agents can also include, but are not limited to agents such as anti-proliferative/antimitotic agents including natural products such as vinca alkaloids (e.g., vinblastine, vincristine, and vinorelbine), paclitaxel, epidipodophyllotoxins (e.g., etoposide and teniposide), antibiotics (e.g., dactinomycin (actinomycin D), daunorubicin, doxorubicin, and idarubicin), anthracyclines, mitoxantrone, bleomycins, plicamycin (mithramycin) and mitomycin, enzymes (e.g., L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; anti-proliferative/antimitotic alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC); anti-proliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine {cladribine}); platinum coordination complexes (e.g., cisplatin and carboplatin), procarbazine, hydroxyurea, mitotane, aminoglutethimide; hormones (e.g., estrogen); anti-coagulants (e.g., heparin, synthetic heparin salts and other inhibitors of thrombin); anti-platelet agents (e.g., aspirin, clopidogrel, prasugrel, and ticagrelor); vasodilators (e.g., heparin, aspirin); fibrinolytic agents (e.g., plasminogen activator, streptokinase, and urokinase), aspirin, dipyridamole, ticlopidine, clopidogrel, abciximab; antimigratory agents; antisecretory agents (e.g., breveldin); anti-inflammatory agents, such as adrenocortical steroids (e.g., cortisol, cortisone, fludrocortisone, prednisone, prednisolone, 6α-methylprednisolone, triamcinolone, betamethasone, and dexamethasone), non-steroidal agents (e.g., salicylic acid derivatives, such as aspirin); para-aminophenol derivatives (e.g., acetaminophen); indole and indene acetic acids (e.g., indomethacin, sulindac, and etodalac), heteroaryl acetic acids (e.g., tolmetin, diclofenac, and ketorolac), arylpropionic acids (e.g., ibuprofen and derivatives), anthranilic acids (e.g., mefenamic acid and meclofenamic acid), enolic acids (e.g., piroxicam, tenoxicam, phenylbutazone, and oxyphenthatrazone), nabumetone, gold compounds (e.g., auranofin, aurothioglucose, and gold sodium thiomalate); immunosuppressives (e.g., cyclosporine, tacrolimus (FK-506), sirolimus (rapamycin), azathioprine, and mycophenolate mofetil); angiogenic agents (e.g., vascular endothelial growth factor (VEGF)), fibroblast growth factor (FGF); angiotensin receptor blockers; nitric oxide donors; anti-sense oligionucleotides and combinations thereof; cell cycle inhibitors, mTOR inhibitors, growth factor receptor signal transduction kinase inhibitors; retinoids; cyclin/CDK inhibitors; HMG co-enzyme reductase inhibitors (statins); and protease inhibitors.

The devices and methods described herein may provide benefits such as modularity that enable various individual device components to be selected and installed together at a treatment site and increase the ability of a physician to adaptably treat an increased range of anatomical variation. Devices in accordance with the present disclosure permit sizing and configuration of elongated segment and/or branch segment components that can conform to the specific geometry of the vasculature at a treatment site.

The devices and methods disclosed herein can provide the physician with a broader range of treatment options as compared to selecting from a limited range of predetermined options. For example, a device in accordance with various embodiments can comprise two elongated segments selected by the physician to provide a combined cross section suitable to approximate the cross section of a vasculature at a treatment site of a patient, and the device may further comprise branch segments that may be added to the elongated segments in a fashion that is more customizable and adapted to the specific needs and anatomy of the patient, with the location at which the branch segment is connected to the elongated segment and the branch segment size determined by the physician based on the anatomy of the patient and with the branch segment added to the device in a modular manner.

The modular nature of devices and systems in accordance with the present disclosure may confer the benefits as described above while reducing the number of separate devices that must be manufactured by a producer or purchased and stocked by a treating facility. The devices and systems of the present disclosed herein may provide the further benefit of reducing the undeployed sizes or diameters of medical devices and the trauma associated with insertion and deployment relative to a treatment device comprising a single component inserted into the region to be treated.

For the avoidance of doubt, the device and methods disclosed herein have been described in the context of providing therapy to the vasculature, however, it should be understood that these devices may be implantable in any suitable body lumen.

Thus, the branched adaptable stent devices and method described herein provides a mechanism to substantially approximate various anatomical configurations of the vasculature or other body lumens, including branch vessel lumens, at a treatment region to minimize leakage around the medical device(s) at the treatment region and isolate a treatment region from fluid pressure.

It will be apparent to those skilled in the art that various modifications and variations can be made in the present disclosure without departing from the spirit or scope of the disclosure. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, materials, elements, components, shape, size and arrangement of parts including combinations within the principles of the invention, to the full extent indicated by the broad, general meaning of the terms in which the appended claims are expressed. To the extent that these various modifications do not depart from the spirit and scope of the appended claims, they are intended to be encompassed therein.

1. A device having a support structure and a covering material, the device operable to be delivered to an at least partially occluded lumen including a non-bifurcated portion, a first bifurcated portion, and a second bifurcated portion, the device comprising:
a first elongated segment having two opposing ends and defining a first primary lumen extending therebetween, the first elongated segment operable to be positioned at least partially in the first bifurcated portion of the partially occluded lumen; and
a second elongated segment having two opposing ends and defining a second primary lumen extending therebetween, the second elongated segment operable to be positioned at least partially in the second bifurcated portion of the partially occluded lumen;
wherein a combined cross section of the first elongated segment and second elongated segment includes a combined cross section that is equal to or greater than an intraluminal cross section of the non-bifurcated portion of the at least partially occluded lumen, the first and second elongated segments having a radial wall strength sufficient to resist inward radial force exerted by the at least partially occluded vessel to resist collapse of the first and second primary lumens.

2. The device of claim 1, wherein the first and second elongated segments are self-expandable.

3. The device of claim 1, wherein the first and second elongated segments are balloon expandable.

4. A device having a support structure and a covering material, the device operable to be delivered to an at least partially occluded lumen including a non-bifurcated portion, a first bifurcated portion, and a second bifurcated portion, the device comprising:
a primary elongated segment having two opposing ends and defining a primary lumen extending therebetween wherein a cross section of the primary elongated segment is equal to or greater than an intraluminal cross section of the non-bifurcated portion of the at least partially occluded lumen;
a first elongated segment having two opposing ends and defining a first secondary lumen extending therebetween, the first elongated segment operable to be positioned at least partially in the first bifurcated portion of the partially occluded lumen; and
a second elongated segment having two opposing ends and defining a second primary lumen extending therebetween, the second elongated segment operable to be positioned at least partially in the second bifurcated portion of the partially occluded lumen of the partially occluded lumen.

5. The device of claim 4, wherein the primary, first, and second elongated segments are self-expandable.

6. The device of claim 5, wherein the primary, first, and second elongated segments are balloon expandable.

7. A device having a support structure and a covering material, the device operable to be delivered to an at least partially occluded lumen including a non-bifurcated portion, a first bifurcated portion, and a second bifurcated portion, the device comprising:
a body including a primary portion, a first branch, and a second branch, the primary portion defining a primary lumen, the primary portion defined between a first open end and a flow divider and having a primary portion length, the first branch defining a first branch lumen, the first branch extending from the primary portion at the flow divider to a first branch open end, the first branch having a first branch length, and the second branch defining a second branch lumen, the second branch extending from the primary portion at the flow divider to a second branch open end, the second branch having a second branch length, the body having a radial wall strength sufficient to resist inward radial force exerted by the at least partially occluded vessel to resist collapse of the primary, first branch, and second branch lumens.

8. The device of claim 7, wherein the body is self-expandable.

9. The device of claim 7, wherein the body is balloon expandable.

10. The device of claim 7, wherein the body length is approximately from 2.5 to 5.5 centimeters.

11. The device of claim 10, wherein the first and second branch lengths are approximately from 2 to 7 centimeters.

12. The device of claim 7, wherein the body includes a diameter from 8 to 24 centimeters.

13. The device of claim 7, wherein the first branch and the second branch include a diameter from 7 to 10 diameters

14. The device of claim 7, further comprising:
a first elongated segment having two opposing ends and defining a lumen extending therebetween, the first elongated segment operable to be positioned at least partially in the first branch lumen; and
a second elongated segment having two opposing ends and defining a lumen extending therebetween, the second elongated segment operable to be positioned at least partially in the second branch lumen.

15. The device of claim 7, wherein a ratio between a length of the first and second branch and the body is about 1:1.
